(19) United States
(12) Patent Application Publication
(10) Pub. No.: US 2005/0235397 A1
(43) Pub. Date: Oct. 27, 2005

(54) Deer tick protection device

(76) Inventor: Paul M. Cosenza, Auburn, MA (US)

Correspondence Address: FISH & RICHARDSON PC, P.O. BOX 1022, MINNEAPOLIS, MN, 55440-1022, US

(21) Appl. No.: 11/167,770

(22) Filed: Jun. 27, 2005

Related U.S. Application Data
(62) Division of application No. 10/083,000, filed on Feb. 26, 2002.
(60) Provisional application No. 60/274,325, filed on Mar. 8, 2001.

(51) Int. Cl. A61F 9/00; A41D 13/08

(52) U.S. Cl. 2/242

(57) ABSTRACT

A device for use during outdoor activities for protection against crawling insects such as deer ticks carrying Lyme Disease includes a set of protection elements. The elements consist of a substrate with an adhesive layer at each surface. A first (inner) adhesive layer is provided for removable attachment of the protection element upon clothing surfaces in regions adjacent to clothing openings where a second (outer) adhesive layer is exposed to impede advancement of crawling insects toward clothing openings, and preferably to securely trap deer ticks that contact the surface. One or both adhesive surfaces may be covered with release sheets removed prior to use. A shield may extend outwardly from the plane of the substrate, generally above the outer adhesive layer, to shield the adhesive surface from the elements, e.g., rain, dew, dust, leaves, grasses, etc., for extending the effective life of the protection element. A method for use of a protection device of the invention during outdoor activities for protection against crawling insects such as deer ticks carrying Lyme Disease is also described.

### TECHNICAL FIELD

This application is a division of U.S. application Ser. No. 10/083,000, filed Feb. 26, 2002, now pending, which claims benefit of U.S. Provisional Patent Application No. 60/274,325, filed Mar. 8, 2001, the complete disclosure of which is incorporated herein by reference.

This invention relates to devices for bodily protection against crawling insects in the outdoors, and, more particularly, against deer ticks carrying Lyme Disease.

### BACKGROUND

In the northeast region of the United States, and in many other regions of the world, the threat of deer ticks carrying Lyme Disease has become an increasing concern for those participating in outdoors activities. Those with particular concerns include walkers and hikers, birdwatchers, hunters, and parents with children. Recommendations by public health officials for precautions against contracting Lyme Disease from deer ticks include, e.g., wearing long pants and shirts with long sleeves, tucking long hair into a cap or hat, tucking shirt tails into pants, placing pant cuffs inside socks or boots, and careful inspection of the body after each exposure. However, deer ticks are extremely small, and the presence of a deer tick may be overlooked even with the most careful inspection.

### SUMMARY

The invention relates to a protection device, and method, for use during outdoor activities for protection against deer ticks carrying Lyme Disease. The device comprises a set of protection elements consisting of a substrate with an adhesive layer at each of the inner and outer surfaces. A first (inner) adhesive layer is adapted for removable attachment of a protection element upon a clothing surface in a region adjacent to, and preferably below, a clothing opening, and a second (outer) adhesive layer is exposed while in use to at least impede the progress of, and preferably to securely trap, deer ticks that contact the adhesive surface, e.g. while crawling on clothing surfaces. One or both of the adhesive surfaces may be covered with a release sheet that is removed prior to use to expose the underlying adhesive surface. A shield may extend generally outwardly from the outer surface of the protection element substrate, generally above the outer adhesive surface, to shield the adhesive from the elements, e.g., rain, dew, dust, leaves, grasses, etc., for extending the effective life of the protection element.

According to one aspect of the invention, a protection device for use during outside activities for protection of a wearer against crawling insects such as deer tick carrying Lyme Disease comprises a set of at least one protection element comprising an elongate substrate having a first, inner surface and an opposite, second, outer surface, a first, inner adhesive layer adhered upon the first, inner surface of the substrate and having an adhesive surface adapted, upon exposure, to releasably adhere the protection element upon a clothing surface in a region adjacent to a clothing opening, and a second, outer adhesive layer adhered upon the second, outer surface of the substrate and having an adhesive surface adapted, upon exposure, to impede advancement of insects crawling upon the clothing surface into contact with the adhesive surface from proceeding thereacross toward a clothing opening.

Preferred embodiments of this aspect of the invention may include one or more of the following additional features. The protection device further comprises an inner release sheet mounted upon the adhesive surface of the first, inner adhesive layer and removable to expose the adhesive surface for adhering the protection element upon a clothing surface. The protection device further comprises an outer release sheet mounted upon the adhesive surface of the second, outer adhesive layer and removable to expose the adhesive surface for at least impeding advancement of the crawling insects across the adhesive surface. In a set of protection elements, the inner release sheet has a first surface removably mounted upon the adhesive surface of the first, inner adhesive layer and an opposite second surface removably mounted upon an opposed adhesive surface of a the second, outer adhesive layer. The set of protection elements comprises multiple protection elements. The set of protection elements comprises at least one protection element sized and configured for adhering upon a clothing surface in a region of at least one clothing opening selected from the following group of clothing openings: waist band, shirt collar, shirt front, shirt sleeves, and pant cuffs. The set of protection elements comprises at least one protection element pre-configured, e.g., for length or shape. The set of protection elements has the form of a roll or the form of a sheet. The protection element further comprises a shield portion extending outwardly from a plane of the substrate relative to and generally above the second, outer adhesive layer. The shield portion is an integral extension of the substrate, e.g., having the form of a curved, outward extension of an edge portion of the substrate or having the form of a protrusion from the second, outer surface of the substrate.

According to another aspect of the invention, a method for protection against insects such as deer ticks carrying Lyme Disease crawling upon a clothing surface of a wearer toward a clothing opening comprises the steps of: a) selecting a protection element from a set of protection elements, the protection element comprising an elongate substrate having a first, inner surface and an opposite, second, outer surface, a first, inner adhesive layer adhered upon the first, inner surface of the substrate and having an adhesive surface, and a second, outer adhesive layer adhered upon the second, outer surface of the substrate and having an adhesive surface; b) exposing the adhesive surface of the first, inner adhesive layer; c) applying the adhesive layer to a clothing surface for removable attachment of the protection element adjacent to a clothing opening; and d) exposing the adhesive surface of the second, outer adhesive layer to impede advancement of insects crawling upon the clothing surface from proceeding across the adhesive surface toward the clothing opening.

Preferred embodiments of this aspect of the invention may include one or more of the following additional features. The method comprises the further step of adhering the protection element generally below a clothing opening to impede advancement of insects crawling generally upward toward the clothing opening. The method comprises the further step of removing a release sheet to expose the adhesive surface of the first, inner adhesive layer. The method comprises the further step of removing a release sheet to expose the adhesive surface of the second, outer adhesive layer, the method further comprising delaying removal of the release sheet to expose the adhesive surface of the second, outer adhesive layer until arriving in a region where protection is desired. The method comprises the further step of adhering a protection element in a region of a clothing opening selected from the following group of clothing openings: waist band, shirt collar, shirt front, shirt sleeves, and pant cuffs. The method further comprises repeating the steps a) through d) to adhere protection elements in regions of multiple clothing openings.

An objective of the invention is to provide a device and method that can be employed easily and effectively during outdoor activities, for protection against deer ticks carrying Lyme Disease.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1 and 2 are somewhat diagrammatic front and partial rear views, respectively, of a person using the protection device of the invention, e.g., during outdoor activities, for protection against crawling insects such as deer ticks carrying Lyme Disease.

FIG. 3 is a plan view of one embodiment of a set of protection elements of the protection device of FIGS. 1 and 2;

FIG. 4 is an end section view of a protection element of the protection device of the invention, taken at the line 4-4 of FIG. 3;

FIGS. 5 and 6 and FIG. 7 are perspective views of other embodiments of protection devices of the invention; and

FIGS. 8, 9, 10, 11 and 12 are end section views of other embodiments of protection elements of protection devices of the invention.

Like reference symbols in the various drawings indicate like elements.

### DETAILED DESCRIPTION

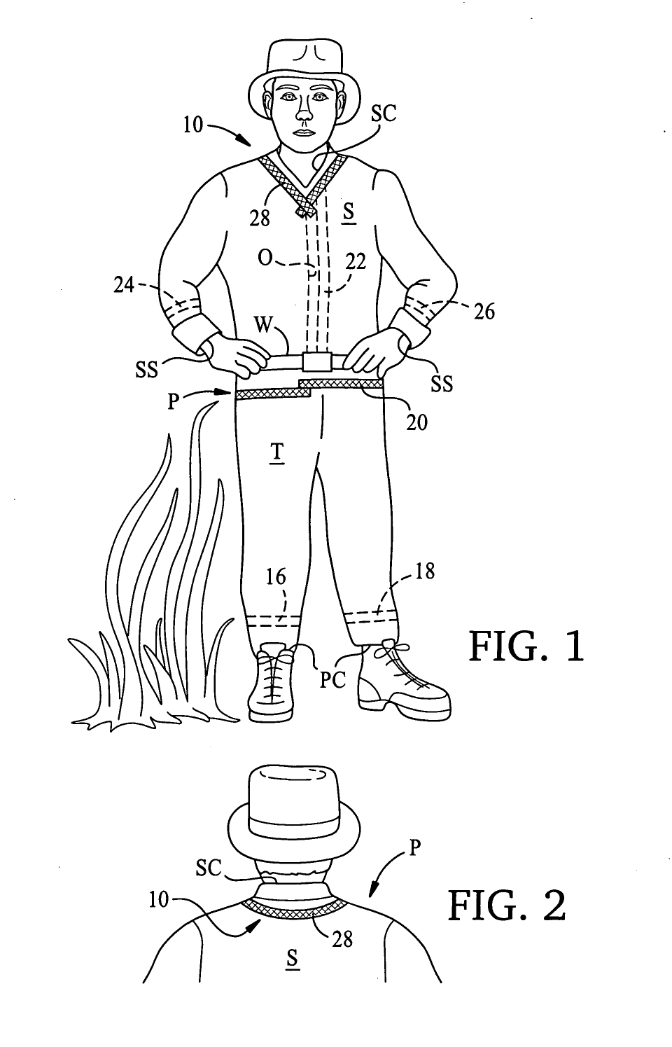

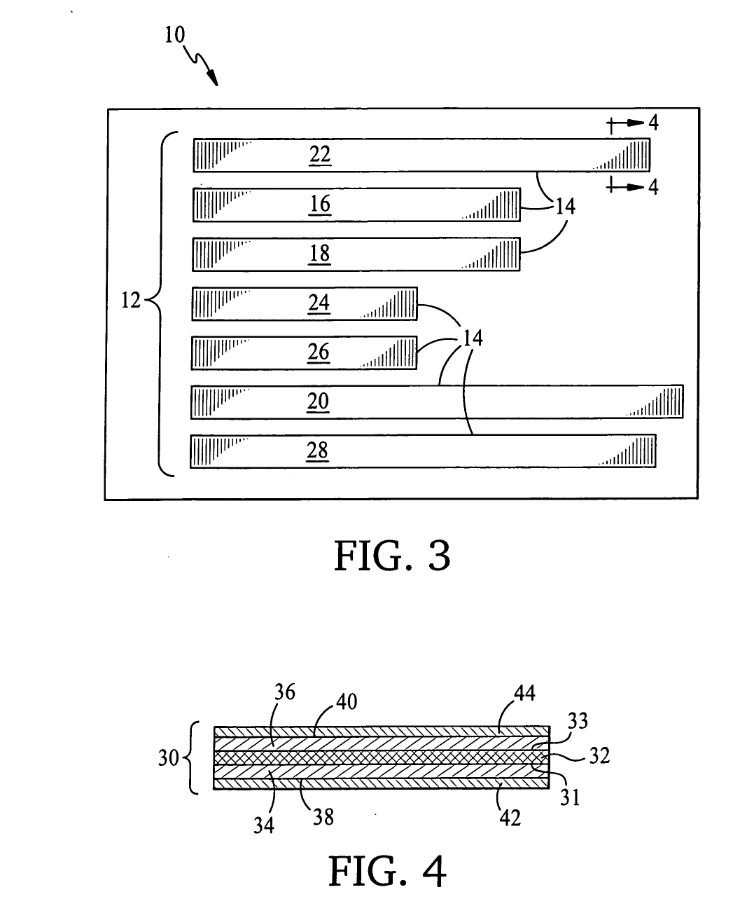

Referring to FIGS. 1 and 2, there is shown a person, P, using a protection device 10 of the invention for outdoor activities, e.g., walking, hiking, hunting, bird watching, playing, etc., for protection against crawling insects such as deer ticks carrying Lyme Disease. Referring now also to FIG. 3, the protection device typically consists of a set 12 of one or multiple protection elements 14 adapted for removable attachment upon the surface of the user's clothing in regions adjacent to, and preferably below, clothing openings to the body. For example, according to one embodiment of the invention, a typical protection device 10 consists of a set 12 of protection elements 14, including at least a pant waist element 20 for attachment to the pants or trousers, T, below and about the waist opening, W; and shirt collar element 28 for attachment to the shirt, S, below and around the shirt collar opening, SC. The set 12 of protection elements 14 may also include one or more of the following (shown in dashed line in FIGS. 1 and 2): pant cuff elements 16, 18 for attachment to the user's pants or trousers, T, in the region of the cuff openings, PC; shirtfront element 22 for attachment to the shirt, S, along the region of the shirtfront opening, O (also shown in dashed line); and shirtsleeve elements 24, 26 for attachment to the shirt, S, about the shirtsleeve cuff openings, SS.

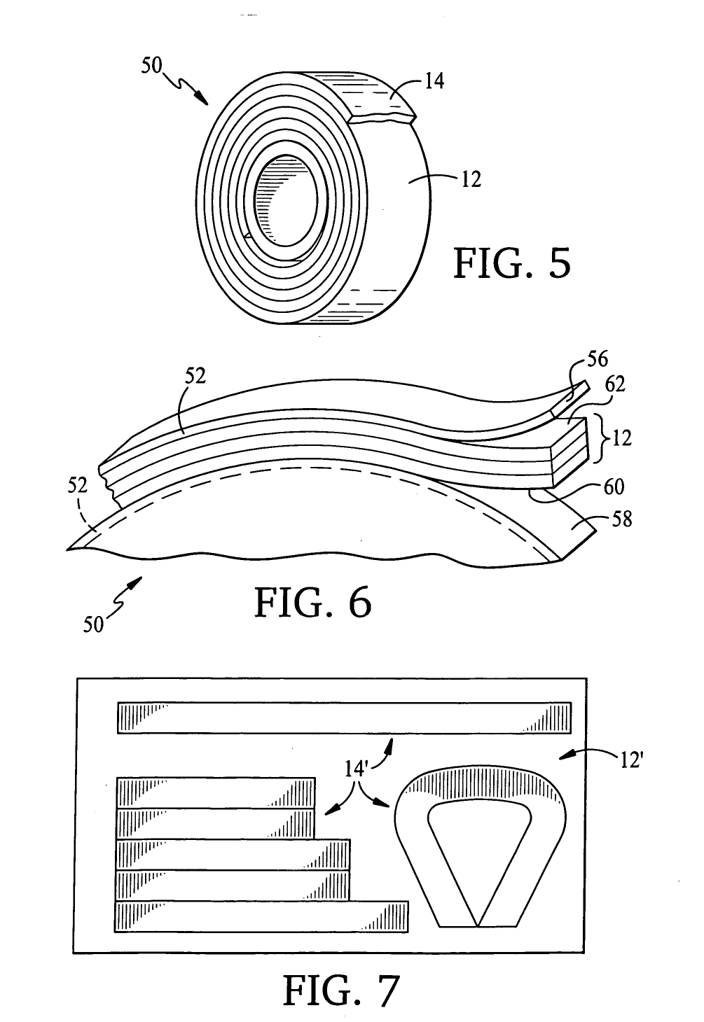

Referring now also FIG. 4, in one embodiment, a protection element 14 has the form of a double-faced adhesive assembly 30 consisting of a thin substrate or carrier 32 with adhesive layers 34, 36 on each of the first, inner and second, outer surfaces 31, 33, respectively. In particular, the adhesive layer 34 defines a first (inner) adhesive surface 38 for removable attachment of the protection element 14 upon the surface of the wearer's clothing, and the adhesive layer 36 defines a second (outer) adhesive surface 40. The adhesive of the outer adhesive layer 36 is formulated to at least impede advancement of insects such as deer ticks crawling upon the clothing surface into contact with the outer adhesive surface 40 from proceeding thereacross toward a clothing opening, and, preferably, the adhesive of the outer adhesive layer is formulated to securely retain and capture deer ticks landing or crawling thereupon. The adhesive of the outer adhesive layer 36 is also preferably formulated to maintain an acceptable degree of tackiness, and therefore protection, for extended periods of time, e.g., for outdoor activities. In preferred embodiments, the adhesive of the outer adhesive layer 36 may also be formulated with a pesticide ingredient to kill deer ticks upon contact with the adhesive.

Referring again to FIG. 4, the protection element 14 may include release or cover sheets 42, 44 upon the inner and outer adhesive surfaces 38, 40, respectively, to protect the adhesive surfaces 38, 40 until the release sheets 42, 44 are removed to expose the adhesive for use. For example, the release sheet 42 upon the inner adhesive surface 38 may be removed at the user's home to allow one or more of the protection elements 14 of the set 12 to be suitably positioned upon the clothing, in advance of reaching the outdoor activities area in which protection is required. For extended effective protection, it is recommended that the release sheet 44 upon the outer adhesive surface 40 be removed only upon reaching the outdoor activities area.

The protection element substrate 32 may be any suitable material, and typically is a polymeric film or tape. The substrate material may clear or lightly translucent, or the substrate 32 and/or one or both adhesive layers 34, 36 may be colored or tinted, e.g., orange, white, gray, camouflage or other color, as desired.

The adhesive material of the inner adhesive layer 34 is preferably formulated for secure attachment of the protection element upon the clothing surface, without gaps, but to permit the protection elements 12 to be removed without damage to the clothing, and without leaving a residue. The adhesive material of the outer adhesive layer 36, and the adhesive material of the inner adhesive layer 34 as well, are preferably formulated to be non-toxic to humans and pets, in normal use. The protection elements 12 and cover sheets 42, 44 are suitable for disposal as regular trash, without hazard to the environment.

The adhesives of the inner and outer adhesive layers 34, 36 may be any suitable adhesive formulation familiar to those skilled in the adhesives art. For example, presently preferred adhesives for the adhesive of the inner adhesive layer 34 and for the adhesive of the outer adhesive layer 36 include synthetic rubber-based adhesives.

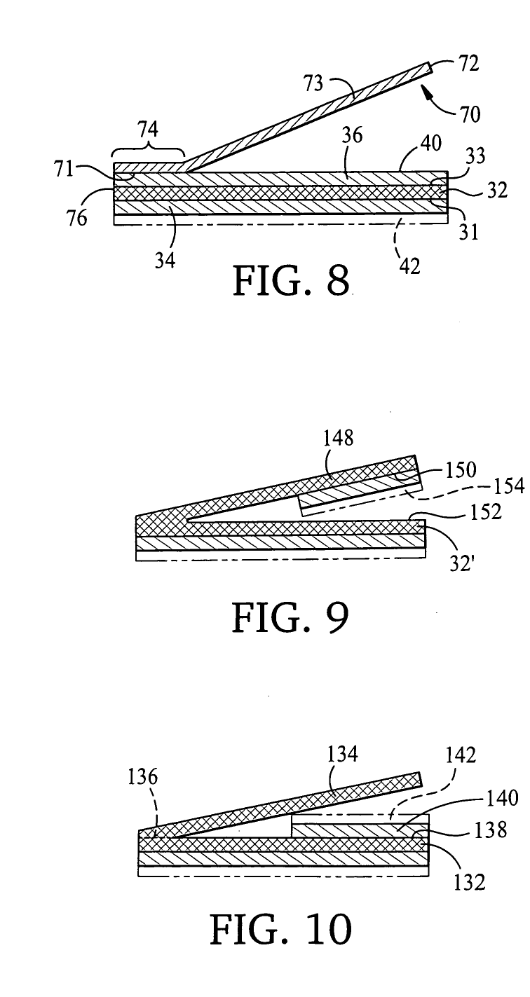

The set 12 of protection elements 14 may be provided is any suitable form, e.g. a roll of tape, other elongated elements, sheets, sets of die cut elements of sizes and/or configurations preferably suitable for intended regions of use, e.g., pant cuffs, waistband, shirtsleeve cuffs, neck or collar, and/or shirtfront, etc. For example, referring to FIG. 5, the set 12 of protection elements 14 may be in the form of a continuous roll of tape 50, from which protection elements 12 of any desired length may be unwound and detached, i.e., cut, from the roll. Referring also to FIG. 6, in this embodiment, a continuous release cover sheet 52 has two release surfaces 56, 58, with the outer surface 58 of an underlying protection element on the roll serving as the release sheet surface for the inner adhesive surface 60 of the next outer-layer of protection elements 12 on the roll. The inner adhesive surface 60 is thus exposed as the protection elements 12 are unwound from the roll 50 while the outer adhesive surface 62 remains covered by the inner surface 56 of the release cover sheet 52 until cover sheet 52 is removed. In other embodiments, (FIGS. 3 and 7), sets 12, 12′ of protection elements 14, 14′ are die-cut and provided in sheet form. In the embodiment described above (with reference to FIG. 3), the protection elements 14 are essentially linear, with a generally uniform width. In another embodiment (FIG. 7), the protection elements 14′ may be specially curved or shaped to conform to intended clothing openings to be protected.

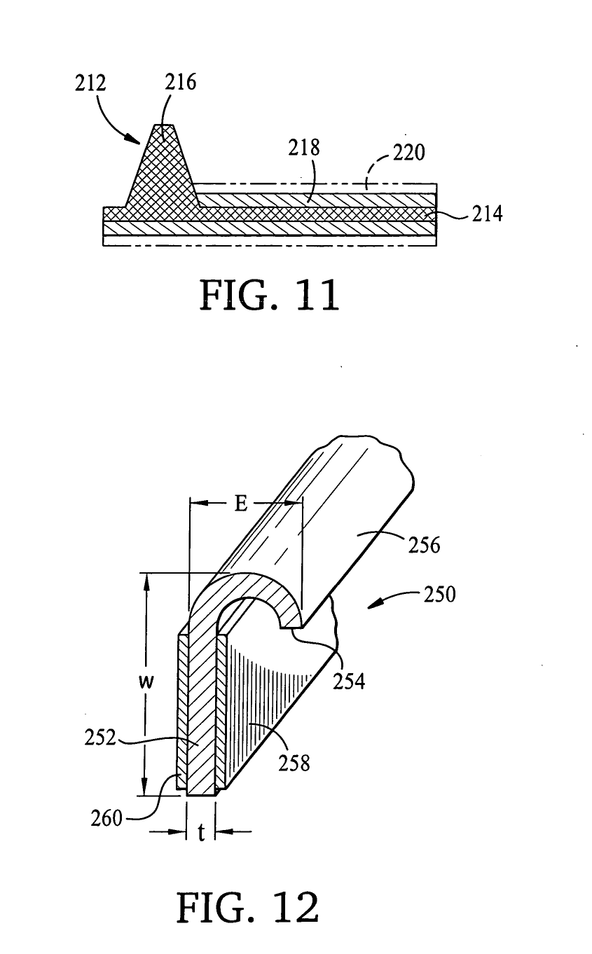

Referring next to FIGS. 8-12, in other preferred embodiments, the protection elements include a flap or shield element disposed and extending generally above the outer adhesive surface. The flap thus provides a degree of shielding for the outer adhesive surface, e.g., against moisture (rain, etc.) or other materials in the field that might otherwise reduce the tackiness, and thereby the effectiveness and/or useful life, of the protection element.

In FIG. 8, a shield element 70 has the form of the cantilevered end 72 of the release cover sheet 73. The cover sheet is secured against complete release, e.g., by a localized reduced coating of release material upon the inner surface 71 of the cover sheet 70, in a region 74 along one edge 76. In this manner, the cover sheet is designed to be preferentially left in place or removed at the option of the user.

Alternatively, in other embodiments, the substrate 32′ may be folded (FIG. 9) or formed of two elements 132, 134 sonic welded or otherwise joined along one edge region, shown in dashed line 136 (FIG. 10). In each of these embodiments, an inner surface 150 of the shield 148 may be coated with an adhesive layer 152, which is covered prior to use with a release sheet 154 (FIG. 9). Alternatively, the outer surface 138 of the main body may coated with an adhesive layer 140, which, again, is protected prior to use with a release sheet 142 (FIG. 10).

In yet another embodiment (FIG. 1), a protection element 212 has a substrate 214 formed, e.g. by molding or extrusion. A shield in the form of a protrusion 216 extends axially and generally above the outer adhesive layer 218. The adhesive surface is protected prior to use by cover sheet 220.

Finally, referring to FIG. 12, in a particular construction of this embodiment of the invention found effective at higher ambient temperatures, a protection element 250 has a substrate 252 of polymeric material, e.g. PVC (poly vinyl chloride), formed by extrusion. The substrate 252 has a width, W, e.g. about 3/4-inch, and a thickness, T, e.g. about 1/16-inch. An upper edge portion 254 of the substrate curves outwardly to extend to a distance, E, e.g., about 1/4-inch, forming a shield 256 for the exposed surface of the outer adhesive layer 258. The outer adhesive layer 258 and the inner adhesive layer 260 are both shown with the adhesive surfaces exposed for use.

A number of embodiments of the invention have been described. Nevertheless, it will be understood that various modifications may be made without departing from the spirit and scope of the invention. For example, the substrate, adhesive and/or release sheet may be colored or otherwise coded at one or both surfaces to enable the user to readily differentiate between intended inner and outer surfaces. The inner and outer adhesive layers may have any formulation(s) found suitable by those skilled in the adhesives art. The adhesives of the inner and outer adhesive layers may be formulated to provide adhesive surfaces of the same or similar tackiness, or of contrasting, i.e. different, tackiness, e.g., the outer adhesive layer may have relatively greater tackiness. Discontinuous protection elements of uniform or differing lengths may be wound upon a roll. Die-cut sets of protection elements may include elements of different widths, e.g., the pant cuff elements may be wider than the shirt cuff elements, due to the level of exposure and desired protection. A set of protection elements provided to or employed by a wearer in the field may differ from the exemplary sets described herein, e.g. in size, configuration, numbers of elements, and/or regions of use upon the wearer's clothing, including due to differences in wearing apparel and/or due to wearer preference. Accordingly, other embodiments are within the scope of the following claims.

1. A method for protection against insects such as deer ticks carrying Lyme Disease crawling upon a clothing surface of a wearer toward clothing openings, said method comprising protection of at least two types of clothing openings selected from the group of types of clothing openings consisting of: waist band, shirt collar, shirt front, shirt sleeves, and pant cuffs by the steps of:
a) selecting a protection element from a set of protection elements comprising an elongate substrate having a first, inner surface and an opposite, second, outer surface, a first, inner adhesive layer adhered upon the first, inner surface of the substrate and having an adhesive surface, and a second, outer adhesive layer adhered upon the second, outer surface of the substrate and having an adhesive surface;
b) exposing the adhesive surface of the first, inner adhesive layer;
c) applying the adhesive surface of the first, inner adhesive layer to a clothing surface to removably attach the protection element to the clothing surface adjacent to a selected type of clothing opening;
d) exposing the adhesive surface of the second, outer adhesive layer to impede advancement of insects crawling upon the clothing surface from proceeding across the adhesive surface of the second, outer adhesive layer, toward the selected type of clothing opening; and
e) repeating the steps a) through d) to adhere protection elements in regions of at least one additional type of clothing opening.

2. The method of claim 1, comprising the further step of adhering the protection element generally below the selected type of clothing opening to impede advancement of insects crawling generally upward toward the selected type of clothing opening.

3. The method of claim 1, comprising the further step of removing a release sheet to expose the adhesive surface of the first, inner adhesive layer.

4. The method of claim 1 or claim 3, comprising the further step of removing a release sheet to expose the adhesive surface of the second, outer adhesive layer.

5. The method of claim 4, further comprising delaying removal of the release sheet to expose the adhesive surface of the second, outer adhesive layer until arriving in a region where protection is desired.

6. A protection system for use during outside activities for protection of a wearer against crawling insects such as deer ticks carrying Lyme Disease, said protection system comprising
a set of protection elements for protection of a corresponding set of clothing openings, with at least one protection element pre-configured to accommodate a correspondingly configured clothing opening, and
a set of clothing articles donned by a person to be protected by the set of protection elements and defining the corresponding set of clothing openings, said set of clothing openings consisting of waist band, shirt collar, shirt front, shirt long sleeves, and long pant cuffs,
each protection element of said set of protection elements comprising:
an elongate substrate having a first, inner surface and an opposite, second, outer surface,
a first, inner adhesive layer adhered upon said first, inner surface of said substrate and having an adhesive surface that, upon exposure, releasably adheres said protection element upon a clothing surface of said set of clothing articles in a region adjacent the corresponding clothing opening, and
a second, outer adhesive layer adhered upon said second, outer surface of said substrate and having an adhesive surface that, upon exposure, acts to impede advancement of insects crawling upon the clothing surface of said set of clothing articles into contact with the adhesive surface of said second, outer adhesive layer from proceeding thereacross toward the corresponding clothing opening.

7. The protection device of claim 6, further comprising an inner release sheet mounted upon the adhesive surface of said first, inner adhesive layer and removable to expose the adhesive surface to adhere said protection element upon the clothing surface.

8. The protection device of claim 6 or claim 7, further comprising an outer release sheet mounted upon the adhesive surface of said second, outer adhesive layer and removable to expose the adhesive surface to at least impede advancement of the crawling insects across the adhesive surface of said second, outer adhesive layer.

9. The protection device of claim 6, further comprising, in said set of protection elements, a release sheet having a first surface removably mounted upon the adhesive surface of said first, inner adhesive layer and removable to expose the adhesive surface of said first, inner adhesive layer to adhere said protection element upon the clothing surface and an opposite second surface removably mounted upon an opposed adhesive surface of said second, outer adhesive layer and removable to expose the adhesive surface of said second, outer adhesive layer to at least impede advancement of the crawling insects across the adhesive surface of said second, outer adhesive layer.

10. The protection device of claim 6, wherein said at least one pre-configured protection element is pre-configured for length.

11. The protection device of claim 6, wherein said at least one pre-configured protection element is pre-configured for shape.

12. The protection device of claim 6, wherein said set of protection elements has the form of a roll.

13. The protection device of claim 6, wherein said set of protection elements has the form of a sheet.

14. The protection device of claim 6, wherein at least one said protection element of said set of protection elements further comprises a shield portion extending outwardly from a plane of said substrate relative to and generally above said second, outer adhesive layer.

15. The protection device of claim 14, wherein said shield portion is an integral extension of said substrate.

16. The protection element of claim 15, wherein said shield portion has the form of a curved, outward extension of an edge portion of said substrate.

17. The protection element of claim 15, wherein said shield portion has the form of a protrusion from the second, outer surface of said substrate.
